(19) United States
(12) Patent Application Publication
(10) Pub. No.: US 2017/0065254 A1
(43) Pub. Date: Mar. 9, 2017

(54) IMAGING AGENT DELIVERY METHOD AND SYSTEM THEREOF

(71) Applicant: National Tsing Hua University, Hsinchu City (TW)

(72) Inventors: Chih-Kuang Yeh, Hsinchu City (TW); Chin-Chou Wu, Hsinchu City (TW)

(21) Appl. No.: 14/846,161

(22) Filed: Sep. 4, 2015

(51) Int. Cl. A61B 8/08; A61M 5/00; A61B 8/00

(52) U.S. Cl. CPC A61B 8/481; A61B 8/5207; A61B 8/54; A61M 5/007

(57) ABSTRACT

An imaging agent delivery method and system thereof are provided. The method includes applying an imaging agent to a region, and alternately performing a heating process and a detecting process to the region with an ultrasound transmitting device and an ultrasound receiving device, respectively. Subsequently, an ultrasound signal acquired is processed by the ultrasound receiving device to obtain a temperature image of the region and a vaporization image of the imaging agent, such that the performance of the imaging agent can be monitored on the basis of the temperature image and the vaporization image.

### BACKGROUND DISCLOSURE

1. Technical Field Disclosure

The present disclosure relates to imaging agent delivery methods, and, more particularly, to an imaging agent delivery method and system for monitoring the imaging agent vaporization mechanism associated with temperature in real-time by using ultrasound equipment.

2. Description of Related Art

Ultrasound technique has been broadly adopted in clinical diagnosis and medical treatment. For example, hyperthermia therapy commonly involves the use of focused ultrasound, in which the body tissue is exposed to slightly higher temperatures to damage and kill cancer cells or to make cancer cells more sensitive to the effects of radiation and certain anti-cancer drugs. In some aspects, hyperthermia therapy can be combined with radiotherapy and chemotherapy to reduce the amount of radiation or chemical dose, so as to improve the treatment quality.

In the hyperthermia therapy using ultrasound, microdroplet is a common drug carrier. When a sinuous wave is applied to the microdroplet by ultrasound, the microdroplet is compressed under a positive pressure and is expanded under a negative pressure. As such, when the negative pressure applied to the microdroplet is large enough, the vapor pressure inside the microdroplet will break the bound of the external phospholipid and result in acoustic droplet vaporization (ADV). Moreover, due to the mismatch between the acoustic impedance and the external media, a strong reflection signal is generated during the transmission of ultrasound waves, which thus allows the microdroplet to serve as an imaging agent. In addition, therapeutic drug can be included in the microdroplet to synchronously perform diagnosis and theranosis.

It can be seen that the release of an ultrasound drug carrier is dependent to the environmental temperature and the negative pressure which induces ADV. However, when such treatment is performed in body tissue, the actual focused distance of ultrasound varies with the tissue attenuation, which causes erroneous heating. Further, if the therapeutic region is limited to a specific area, the prior art cannot predict and control the releasing position of the drug and the amount of dose to be released.

Therefore, how to find a simple and efficient method to solve the abovementioned problems without significantly modifying the currently available equipment becomes the objective being pursued by persons skilled in the art.

SUMMARY OF THE DISCLOSURE

Given above-mentioned defects of the prior art, the present invention provides an imaging agent delivery system using ultrasound. With such a system, the released position of the drug and the amount of dose to be released can be monitored and controlled.

In order to achieve above-mentioned and other objectives, the present invention provides an imaging agent delivery system, comprising: an ultrasound transmitting device performing a heating process to a region that an imaging agent is applied, an ultrasound receiving device performing a detecting process to the region to acquire an ultrasound signal, a controller alternately operating the ultrasound transmitting device and the ultrasound receiving device, and an image processing unit, processing the ultrasound signal to obtain a temperature image of the region and a vaporization image of the imaging agent.

In an embodiment, the imaging agent includes a thermal-sensitive imaging agent and a therapeutic drug having fluorocarbons-containing material.

The present invention further provides an imaging agent delivery method, comprising: applying an imaging agent to a region, performing a heating process to the region with an ultrasound transmitting device, performing a detecting process to the heated region with an ultrasound receiving device to acquire an ultrasound signal, processing the ultrasound signal to obtain a temperature image of the region and a vaporization image of the imaging agent, and monitoring vaporization mechanism of the imaging agent based on the temperature image and the vaporization image, wherein the heating process and the detecting process are alternately performed.

In an embodiment, the method further comprises predicting the performance of the imaging agent by performing a preheating process prior to applying the imaging agent.

In another embodiment, the imaging agent includes a thermal-sensitive imaging agent and a therapeutic drug, and the thermal-sensitive imaging agent includes fluorocarbons-containing material.

According to the prior art, since the ultrasound system can only perform heating and detecting individually, releasing position of the drug and the amount of dose to be released cannot be predicted and controlled. By contrast, the present invention provides an imaging agent delivery method and system for monitoring the imaging agent vaporization mechanism associated with temperature in real-time by using ultrasound equipment, releasing position of the drug and the amount of dose to be released are thus predictable and can be controlled. In addition, the imaging agent delivery method and system according to the present invention require no significant modification of the conventional ultrasound equipment, such that the imaging agent delivery method and system of the present invention are compatible with the conventional ultrasound system.

### BRIEF DESCRIPTION OF DRAWINGS

The present invention can be more fully understood by reading the following detailed description of the exemplary embodiments, with reference made to the accompanying drawings, wherein:

FIG. 1 is a system structure view of an imaging agent delivery system according to the present invention;

FIG. 2 is a comparison chart showing the relationship between acoustic droplet vaporization (ADV) efficiency and ADV acoustic pressure at temperatures from 37° C. to 41° C.;

FIG. 3 is a flow chart of image processing performed by an image processing unit of the imaging agent delivery system according to the present invention;

FIGS. 4A-4C are scheme views of a process for obtaining an ADV difference image by subtracting a pre-image before ADV from a post-image after ADV;

FIG. 5 is a scheme view of the ADV difference image obtained in the process shown in FIG. 4 overlaying on a temperature image obtained in the method illustrated in FIG. 2; and

FIG. 6 is a bar diagram of the overlaying percentages under different acoustic pressures at temperatures of 40° C. and 41° C.

### DETAILED DESCRIPTION OF THE DISCLOSURE

In the following, specific embodiments are provided to illustrate the detailed description of the present invention. Persons skilled in the art can easily conceive the other advantages and effects of the present invention, based on the disclosure of the specification. The present invention can also be carried out or applied by other different embodiments.

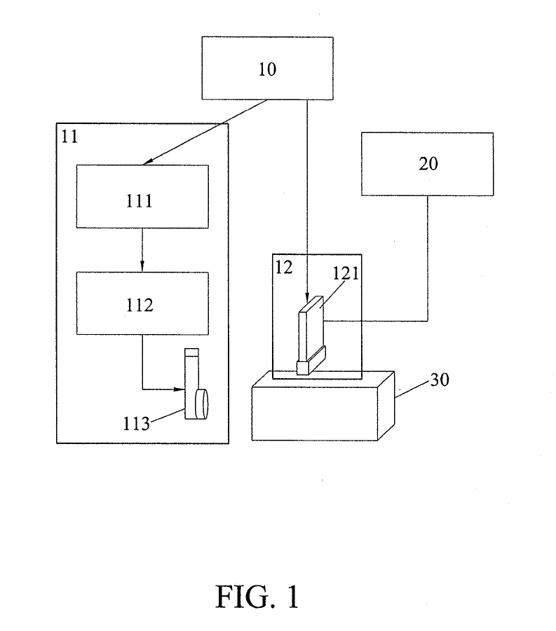

As shown in FIG. 1, a system structure view of an imaging agent delivery system 1 according to the present invention is provided. The imaging agent delivery system 1 includes an ultrasound transmitting device 11, an ultrasound receiving device 12, a controller 10, and an image processing unit 20, so as to monitor and control the imaging agent delivery.

In the operation of the imaging agent delivery system 1, the ultrasound transmitting device 11 may first optionally perform a preheating process before an imaging agent is applied, such that a region to be heated of a subject 30 can be predicted and accordingly adjusted based on the result of the preheating process. Subsequently, the ultrasound transmitting device 11 may perform a heating process to the region of the subject 30 after the imaging agent is applied, and the ultrasound receiving device 12 may perform a detecting process to the region of the subject 30 to acquire an ultrasound signal. Moreover, the controller 10 is capable of alternately operating the ultrasound transmitting device 11 to accelerate the acoustic droplet vaporization (ADV) mechanism of the imaging agent at the heated region and operating the ultrasound receiving device 12 to acquire the ultrasound signal of the subject 30, such that the ultrasound receiving device 12 is prevented from receiving disturbance generated by the ultrasound transmitting device 11. After the ultrasound signal is acquired by the ultrasound receiving device 12, the image processing unit 20is utilized to demodulate the ultrasound signal to obtain a temperature image and an ADV image. In an embodiment, the image processing unit is configured to overlay the temperature image and the vaporization image

In an embodiment, the imaging agent includes a thermal-sensitive imaging agent, such as perfluoropentane (C₅F₁₂), and a therapeutic drug. As such, the ADV of the imaging agent represents the release of the therapeutic drug.

In an embodiment, the ultrasound transmitting device 11 includes a signal generator 111, a power amplifier 112 and a high-intensity ultrasound probe (or a focused ultrasound probe) 113, where the power amplifier 112 is configured to amplify signals of the signal generator 111 and transmitting the amplified signals to the high-intensity ultrasound probe 113. Also, in the embodiment, the ultrasound receiving device 12 includes a linear array of probes (or imaging probes) 121. The high-intensity ultrasound probe 113, for example, has a diameter of 3.3 cm, a radius of curvature of 3.5 cm, and a center frequency of 3.5 MHz, and the linear array of probes 121, for example, has a center frequency of 7.5 MHz. However, persons skilled in the art should appreciate that the high-intensity ultrasound probe and the linear array of probes of the present invention can be selected as any appropriate ultrasound probe and are not limited thereto.

In an embodiment, the controller 10 is established by LabVIEW (National Instruments Corporation, Austin, Tex., USA) platform, where the signal generator 111 of the ultrasound transmitting device 11 is connected with the controller 10 via an IEEE 488 interface, and the ultrasound receiving device 12 is connected with the controller 10 via an IEEE 1394A interface. However, persons skilled in the art should appreciate that the controller of the present invention can be established by any appropriate programming platform, and is thus not limited thereto.

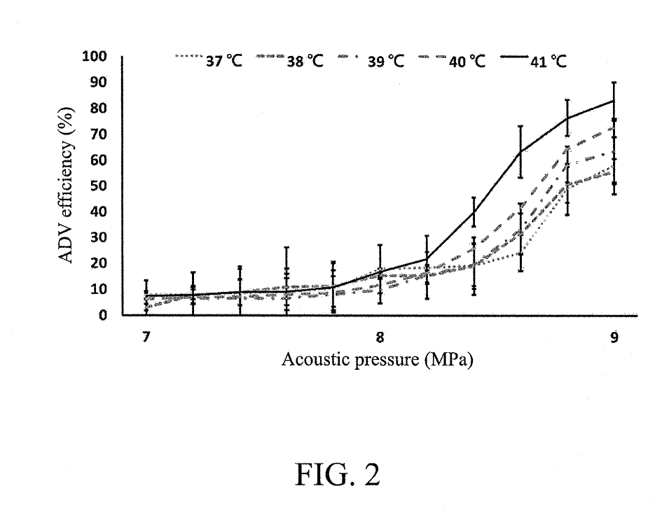

FIG. 2 is a comparison chart showing the relationship between ADV efficiency and ADV acoustic pressure at temperatures from 37° C. to 41° C. In this comparison chart, the ADV efficiency is determined in an ADV experiment with the equation as follows:

$$ADV efficiency ( \% ) = \frac{\sum number ( experimental group )}{\sum number ( control group )} \times 100 \%$$

In the ADV experiment, a syringe pump is utilized to pump the imaging agent flowing through a certain region, and the waste is collected and analyzed by a particle size analyzer to measure the number of vaporized particles, where the ultrasound signal is applied to the certain region for inducing the occurrence of ADV in the experimental group, and no ultrasound signal is applied in the control group. With such experiment and the comparison chart of FIG. 2, an appropriate combination of the ADV acoustic pressure and temperature for achieving desired ADV efficiency is known, and the ultrasound transmitting device 11 of the imaging agent delivery system 1 can operate accordingly to achieve desired ADV efficiency. However, persons skilled in the art should appreciate that the ultrasound transmitting device 11 can operate under any appropriate combination of the ADV acoustic pressure and temperature, and is not limited thereto.

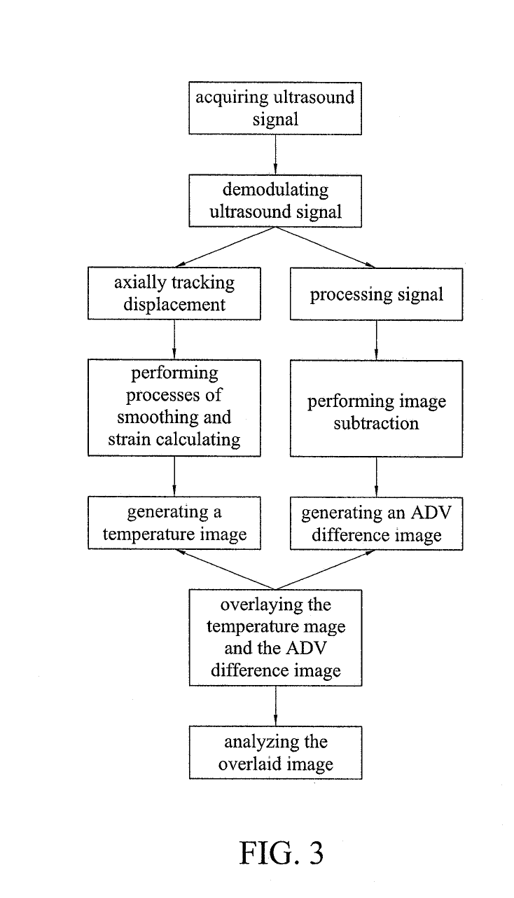

FIG. 3 is a flow chart of image processing performed by the image processing unit 20 of the imaging agent delivery system 1. As illustrated, the ultrasound signal acquired by the ultrasound receiving device 12 is processed by the image processing unit 20, and is demodulated. Subsequently, the demodulated signal is individually processed by two parts.

In the first part, the demodulated signal is analyzed by a speckle tracking method using one-dimensional windows of different sizes to obtain a displacement having largest correlation coefficient, and then the displacement difference of two-dimensional distribution is obtained by axially tracking the displacement. After the displacement difference of two-dimensional distribution is corrected, the processes of smoothing and strain calculating are performed to calculate an actual temperature image based on the equation

$$Δ T ( z ) = k \frac{\partial }{\partial z} ( Δ d ) .$$

where ΔT(z) is an accumulated value of temperature changes, k is a constant, Δd is an accumulated value of displacement, and ∂/∂z(Δd) is the amount of thermal strains.

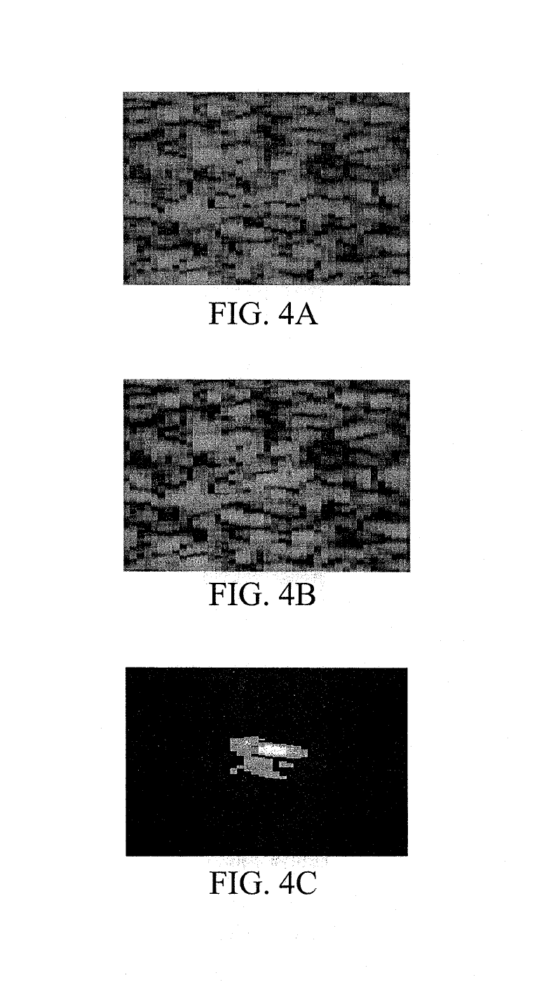

In the second part, the demodulated signal is processed to obtain an ultrasound image. As shown in FIGS. 4A-4C, in order to eliminate the background noise, a pre-image before ADV illustrated in FIG. 4A is subtracted from a post-image after ADV illustrated in FIG. 4B, and an ADV difference image illustrate in FIG. 4C is thus obtained.

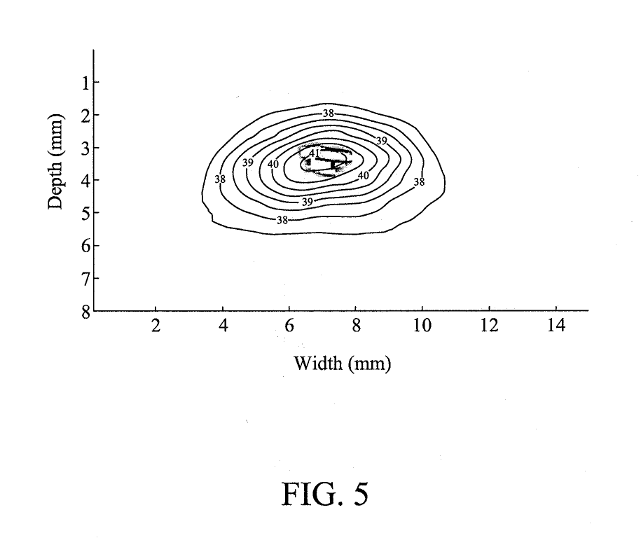

After the temperature image and the ADV difference image are both generated, the image processing unit 20 overlays the ADV difference image and the temperature image to obtain an overlaid image. Accordingly, the overlaid image can be analyzed to monitor whether the ADV occurs in a desired region. FIG. 5 illustrates an exemplary overlaid image, and the overlaid image includes temperature contours from 38° C. to 41° C. and the ADV difference image overlapping the central contours, which intuitively shows that the occurrence of ADV matches the region heated by the ultrasound transmitting device 11.

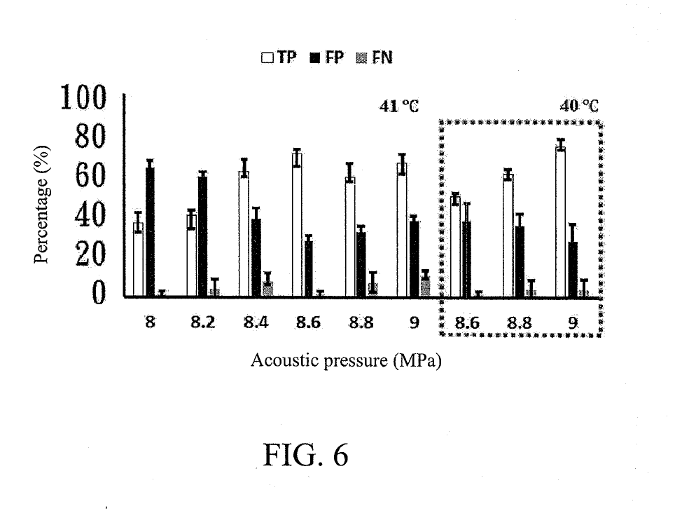

In addition, the overlaid image can also be analyzed with Boolean logic, such that a bar diagram of overlaying percentages can be obtained. FIG. 6 shows an exemplary bar diagram of overlaying percentages under different acoustic pressures at temperatures of 40° C. and 41° C., where TP bar represents true positive, i.e., the percentage that the heated region has ADV, FP bar represents false positive, i.e., the percentage that the heated region has no ADV, and FN bar represents false negative, i.e., the percentage that a non-heated region has ADV. In the exemplary bar diagram of FIG. 6, two scenarios at different temperatures of 40° C. and 41° C. are provided to show that the desirable percentage of TP bar can be achieved at different temperatures by adjusting the acoustic pressure. For example, the TP bar is above 60% under the acoustic pressure of 8.6 MPa at the temperature of 41° C. When it is at the temperature of 40° C., although the TP bar is below 60% under the acoustic pressure of 8.6 MPa, the TP bar is above 60% when the acoustic pressure is increased to 9 MPa. Persons skilled in the art should appreciate that although only temperatures of 40° C. and 41° C. are shown in the exemplary bar diagram, any other suitable temperature can also be selected to accomplish the present invention.

The present invention also provides an imaging agent delivery method similar to the operation of the imaging agent delivery system as mentioned above. Specifically, a preheating process may be optionally performed prior to applying an imaging agent to predict the performance of the imaging agent. After the imaging agent is applied to a region of the target 30, a heating process is performed to the region with an ultrasound transmitting device 11, and a detecting process is performed to the heated region with an ultrasound receiving device 12 to acquire an ultrasound signal. In an embodiment, the heating process and the detecting process are alternately performed. After the ultrasound signal is acquired, a temperature image of the region and a vaporization image of the imaging agent can be accordingly obtained, for example, by a process of displacement correction, smoothing, and strain calculation as mentioned above, such that the vaporization mechanism of the imaging agent can be monitored based on the temperature image and the vaporization image. For example, the vaporization mechanism of the imaging agent can be monitored by overlaying the temperature image and the vaporization image.

In an embodiment, the ultrasound transmitting device 11 and the ultrasound receiving device 12 are controlled by a controller 10, and the heating process and the detecting process are performed alternately. The controller 10 controls the ultrasound transmitting device 11 to accelerate the vaporization mechanism of the imaging agent by heating at the region of the subject 30.

In an embodiment, the imaging agent includes a thermal-sensitive imaging agent, such as fluorocarbons-containing materials, and a therapeutic drug. As such, the ADV of the imaging agent represents the release of the therapeutic drug.

In an embodiment, the ultrasound transmitting device 11 includes a signal generator 111, a power amplifier 112 and a high-intensity ultrasound probe 113.The power amplifier 112 is configured to amplify signals of the signal generator 111, and transmitting the amplified signals to the high-intensity ultrasound probe 113. In an embodiment, the ultrasound receiving device 12 includes a linear array of probes 121. The high-intensity ultrasound probe 113, for example, preferably has a diameter of 3.3 cm, a radius of curvature of 3.5 cm, and a center frequency of 3.5 MHz, and the linear array of probes 121, for example, has a center frequency of 7.5 MHz. However, persons skilled in the art should appreciate that the high-intensity ultrasound probe and the linear array of probes of the present invention can be selected as any appropriate ultrasound probe and are not limited thereto.

From the foregoing, the present invention provides an imaging agent delivery method and system for monitoring the imaging agent vaporization mechanism associated with temperature in real-time by using ultrasound equipment, releasing position of the drug and the amount of dose to be released are thus predictable and can be controlled. In addition, the imaging agent delivery method and system of the present invention require no significant modification of the conventional ultrasound equipment, such that the imaging agent delivery method and system of the present invention are compatible with the conventional ultrasound system.

The above examples are only used to illustrate the principle of the present invention and the effect thereof, and should not be construed as to limit the present invention. The above examples can all be modified and altered by persons skilled in the art, without departing from the spirit and scope of the present invention as defined in the following appended claims.

What is claimed is:

1. An imaging agent delivery system, comprising:
an ultrasound transmitting device performing a heating process to a region that an imaging agent is applied;
an ultrasound receiving device performing a detecting process to the region to acquire an ultrasound signal;
a controller controlling the ultrasound transmitting device and the ultrasound receiving device; and
an image processing unit processing the ultrasound signal to obtain a temperature image of the region and a vaporization image of the imaging agent.

2. The imaging agent delivery system of claim 1, wherein the region is adjusted based on performing a preheating process before the imaging agent is applied.

3. The imaging agent delivery system of claim 1, wherein the imaging agent includes a thermal-sensitive imaging agent and a therapeutic drug.

4. The imaging agent delivery system of claim 3, wherein the thermal-sensitive imaging agent includes fluorocarbons-containing material.

5. The imaging agent delivery system of claim 3, wherein the controller controls the ultrasound transmitting device to accelerate a vaporization mechanism of the imaging agent at the heated region.

6. The imaging agent delivery system of claim 1, wherein the image processing unit overlays the temperature image and the vaporization image.

7. The imaging agent delivery system of claim 1, wherein the temperature image is obtained by a process of displacement correction, smoothing, and strain calculation.

8. The imaging agent delivery system of claim 1, wherein the ultrasound transmitting device includes a focused ultrasound probe, and the ultrasound receiving device includes imaging probes.

9. A method, comprising:
applying an imaging agent to a region;
performing a heating process to the region with an ultrasound transmitting device;
performing a detecting process to the heated region with an ultrasound receiving device to acquire an ultrasound signal, wherein the heating process and the detecting process are alternately performed;
processing the ultrasound signal to obtain a temperature image of the region and a vaporization image of the imaging agent; and
monitoring a vaporization mechanism of the imaging agent based on the temperature image and the vaporization image.

10. The method of claim 9, further comprising predicting the performance of the imaging agent by performing a preheating process prior to applying the imaging agent.

11. The method of claim 9, wherein the imaging agent includes a thermal-sensitive imaging agent and a therapeutic drug.

12. The method of claim 11, wherein the thermal-sensitive imaging agent includes fluorocarbons-containing material.

13. The method of claim 11, wherein the ultrasound transmitting device and the ultrasound receiving device are controlled by a controller to alternately perform the heating process and the detecting process, and the controller controls the ultrasound transmitting device to accelerate the vaporization mechanism of the imaging agent by heating at the region.

14. The method of claim 9, wherein the monitoring further comprises overlaying the temperature image and the vaporization image.

15. The method of claim 9, wherein the temperature image is obtained by a process of displacement correction, smoothing, and strain calculation.

16. The method of claim 9, wherein the ultrasound transmitting device includes a focused ultrasound probe, and the ultrasound receiving device includes imaging probes.
